(19) United States
(12) Patent Application Publication
(10) Pub. No.: US 2010/0297747 A1
(43) Pub. Date: Nov. 25, 2010

(54) SYSTEM AND METHOD FOR MONITORING CELL GROWTH

(76) Inventors: Scott R. Manalis, Cambridge, MA (US); Andrea K. Bryan, Allston, MA (US); Yao-Chung Weng, Cambridge, MA (US); Thomas Burg, Cambridge, MA (US); William H. Grover, Medford, MA (US); Marc W. Kirschner, Newton, MA (US); Paul Jorgensen, Cambridge, MA (US); Michel Godin, Beaconsfield (CA)

Correspondence Address: CHOATE, HALL & STEWART LLP, TWO INTERNATIONAL PLACE, BOSTON, MA, 02110, US

(21) Appl. No.: 12/739,509

(22) PCT Filed: Oct. 24, 2008
(86) PCT No.: PCT/US08/81095

§ 371 Date: Aug. 9, 2010

Related U.S. Application Data
(60) Provisional application No. 60/982,506, filed on Oct. 25, 2007.

(51) Int. Cl. C12M 1/34

(52) U.S. Cl. 435/287.3

(57) ABSTRACT

Microsystem for monitoring cell growth. A microfluidic structure is designed to allow cells to circulate therethrough and the microfluidic structure includes modules to monitor mass, mass density and fluorescence of the cell.

This application claims priority to provisional application Ser. No. 60/982,506 filed Oct. 25, 2007, the contents of which are incorporated herein in their entirety.

This invention arose pursuant to NIH Grant P50GM68762. The Government has certain rights in the invention.

### BACKGROUND OF THE INVENTION

This invention relates to method and system for monitoring cell growth and more particularly to monitoring mass, mass density and fluorescence of single cells in microfluidic systems.

Cell size lies at the nexus of two core cell processes that have been subjected to considerable scrutiny: cell growth and the cell division cycle. Care must be given in considering the relationship between cell size, cell growth, and the cell cycle [2]. The numbers in brackets refer to the references appended hereto. Cell size is a catchall descriptor that, depending on the context, can refer to linear cell dimensions, cell volume, or cell mass. Cell mass is often of most concern. Linear dimensions and volume can change by rearranging the cytoskeleton or altering ion balance, whereas cell mass changes reflect more fundamental events in metabolism and thus are considered to be a direct measurement of cell growth. Accumulating mass requires investments of cellular energy and the acquisition of the requisite small molecule building blocks (e.g. amino acids) to allow a net synthesis of macromolecules. Mass decreases when cells divide or undergo autophagy (i.e. “self-eating”).

Accurate measurements of cell size are fundamental to understanding the size homeostasis of proliferating and resting cells. Specifically for the cell cycle, exponentially growing cells require coordination between cell growth and division to maintain time invariant size distribution of the population in steady state, but it remains unclear how individual cells monitor and translate their size into a signal for cell cycle progression or cell division. The key to cell cycle control is the concentration of critical regulatory proteins, which is defined not only by expression levels, but also by the volume of the cell. By modulating the mass to volume ratio, the cell density, cells may regulate cell cycle events. This possibility remains poorly explored mainly due to lack of tools for directly and accurately measuring cell mass and density. Cell volume is measured via microscopy or the resistive (Coulter) method, but volume is influenced by the chemical environment and does not necessarily detail changes in cell mass. Density measurements offer a cell size index that accounts for changes in either mass or volume, or both. Direct and high-throughput density measurements could assess cell growth in a variety of applications, such as cell cycle control and response to changes in the cell's chemical and physical environment. Aside from cell state measurements, density offers a means to identify specific cell types in order to count, and ultimately sort, by cell type and state.

Density measurements have been limited to density gradient centrifugation and sedimentation, or a combination of indirect mass and volume measurements. In density gradients, cell populations must be large and the density of the cell may be artificially altered by the chemicals in the gradient medium. On a continuous gradient, quantifying the density distribution requires centrifugation to equilibrium and gradient fractionation from which just a few hundred cells are counted. There is no acceptable method for measuring cell density, and what is required is absolute quantification of the density of cell populations with minimal sample perturbation and with a means to collect measured samples for additional studies.

It is therefore an object of the present invention to provide a microsystem for cell sizing (MCS) that will, in a single step, overcome technical limitations that have stifled research into the classic problem in cell biology of how cells control their size.

### SUMMARY OF THE INVENTION

In one aspect, the microsystem for monitoring cell growth according to the invention includes a microfluidic structure to circulate cells in constant order, the microfluidic structure including modules to monitor mass, mass density and fluorescence of the cell. In one embodiment, the microsystem includes a microfluidic rotary channel that allows cells to circulate therethrough in a single file. Several techniques can be used to maintain cell order inside the rotary channel. The channel can be sized to prevent one cell from passing another. Plugs of an immiscible fluid (e.g. oil in water) or phase (e.g. air in water) can also be used to separate or compartmentalize the cells. In addition, inertial effects can be used to focus and order a stream of cells inside channels with asymmetric turns [Di Carlo et al. Continuous inertial focusing, ordering, and separation of particles in microchannels. Proc Natl Acad Sci USA (2007) vol. 104 (48) pp. 18892-7]. A microfluidic pump circulates the cells through the rotary channel and a fluid delivery module delivers nutrients and analytes within the rotary channel. A fluorescence module monitors fluorescence from the cell and a volume detection module determines volume of the cell. A mass detection module is provided for determining mass of a cell and a mass density detection module is provided for determining mass density of the cell. Additional modules can be included in the rotary to measure other properties of the cell.

In another aspect, the microsystem for monitoring cell growth according to the invention includes a microfluidic structure to circulate cells in random order (with or without a need to maintain single file), the microfluidic structure including modules to monitor mass, mass density and fluorescence of the cell. Since the SMR can resolve the mass of mammalian cell to 0.01%, the measured mass of each cell would serve as an effective ‘barcode’ for registration. This approach should be feasible provided that there aren't too many cells in the loop (we estimate 10-100 cells).

In yet another aspect, the microsystem for monitoring cell growth according to the invention includes a means for moving the same collection of cells back and forth through a particular module so that each cell can be measured multiple times during growth. For example, a capillary containing 10-100 cells spaced by at least 100 microns apart is attached to the input port of the suspended microchannel resonator. The capillary is pressurized so that the cells flow through the resonant microchannel one-by-one and are then collected in a second capillary that is attached to the output. Next, the second capillary is pressurized and the cells return through the resonator as the mass of each cell is measured for the second time. This process is continually repeated with automated pressure control devices throughout the growth cycle. The cells can either remain in single-file order, or they could be in random order and be registered by their mass. For a second example, a variation of this approach is used to repeatedly measure a single cell as it flows back and forth within the suspended microchannel resonator. In this case, the cell of interest stays in close proximity to the suspended microchannel and does not traverse through capillaries or other modules.

In a preferred embodiment, the mass detection module includes a suspended microchannel resonator. In a preferred embodiment, the volume detection module includes a cell volume measurement based on the Coulter Principle. The suspended microchannel resonator may include an optical trap for manipulating a cell.

### BRIEF DESCRIPTION OF THE DRAWING

FIG. 1 is a schematic illustration of an embodiment of the invention.

FIG. 2 is a perspective view of a suspended microchannel resonator.

### DESCRIPTION OF THE PREFERRED EMBODIMENT

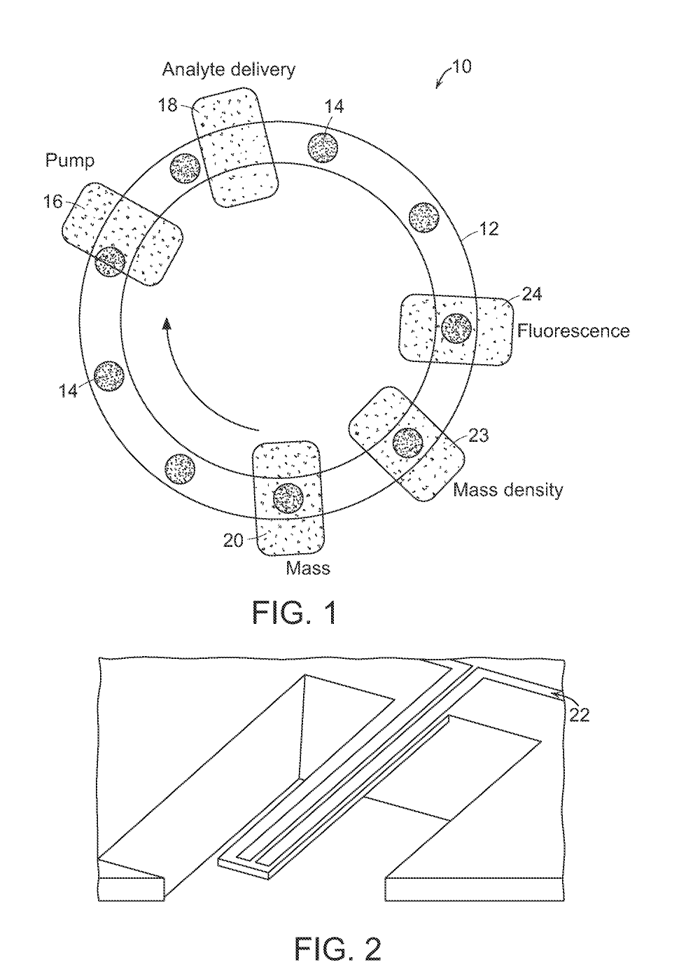

With reference first to FIG. 1, a microsystem 10 includes a microfluidic rotary structure 12 through which cells 14 travel in single file. The cells are circulated by means of a pump 16 and analyte delivery module 18 delivers nutrients and analytes into the rotary structure 12. As shown in FIG. 1, the cells 14 pass several modules that detect mass, mass density, fluorescence, and other properties of the cells.

Still referring to FIG. 1, a mass detector 20 is a suspended microchannel resonator 22 shown in FIG. 2 [1]. The resonance frequency of a suspended microchannel resonator (SMR) is highly sensitive to the presence of cells whose mass density differs from that of the solution. Cells in solution flow through the resonator 22 and its resulting frequency shift depends on their mass and position within the channel. For dilute suspensions, this measurement yields a series of well-separated peaks whose heights are directly proportional to the mass excess of each cell in solution. A low flow rate enables higher-resolution frequency measurements by increasing the transit time of the cell through the device. As described in [1], we can weigh several hundred cells individually in a few minutes with a femtogram resolution and produce a histogram of cell masses.

Continuing to refer to FIG. 1, element 24 is a fluorescence detection module. Microfluidic flow cytometers have been previously demonstrated by several laboratories [22]. We will adopt a similar methodology for our first generation systems. External excitation for conventional reporters (e.g. GFP and RFP) or immunostains will be aligned and focused in the microchannel 12 and external optics will be provided for collecting the resulting signal. However, since mass and density detection with the SMR requires ˜1 second per cell, our fluorescent readout will be considerably slower than conventional flow cytometry. In later systems, the additional measurement time will be used to increase the intensity resolution and thereby enable detection and localization of reporters at low concentrations. As cells move through the system at very slow speeds relative to conventional FACS machines, it will be possible to resolve spatial localization of fluorescence in addition to intensity. To this end, fluorescence peaks can be recorded as cells pass through a focused excitation beam, or an imaging system could be used to distinguish nuclear, cytoplasmic, and plasma membrane localization. Many signaling mechanisms cause cytoplasmic proteins to accumulate in the nucleus or at the plasma membrane (and vice versa).

The pump module 16 is an integrated microfluidic pump and may be a monolithic membrane pump. The analyte delivery module 18 may consist of one or more monolithic membrane “bus valves.” Among microfluidic valves, these three-way bus valves are particularly well suited for adding fluid to and removing fluid from a rotary channel [Paegel et al. Microfluidic serial dilution circuit. Anal Chem (2006) vol. 78 (21) pp. 7522-7].

Referring still to FIG. 1, a mass density module 23 detects the mass density of the cell. Those of skill in the art will recognize that cell density can also be calculated from a measurement of cell mass if cell volume can be determined. It is preferred that the Coulter Principle be used to measure cell volume. In one embodiment, electrical current through the SMR is monitored as the cell flows through it thereby enabling the cell's volume and mass to be measured simultaneously. In another embodiment, current is measured through a channel that is separate from the SMR. This channel is designed and optimized for the Coulter Principle. Another approach for measuring volume is the volume exclusion method (VEM) to measure the rate of change and absolute volume of growing cells at a single cell level. A volume exclusion method device must meet the metabolic needs of the cell. This requirement demands that at least one of the device surfaces be gas permeable or that fresh media is constantly introduced. If the system is in constant flow, then evaporation through this permeable surface will be negligible.

The cell must be kept in media for the majority of the experiment and there must be means for temperature control of the fluid. The cell may be measured multiple times in order to improve precision. One approach to this requirement is to pass the cell back and forth through a fluorescent dye sensing zone or to cycle the cell through the sensing zone. Another approach is to set up a method of continuous measurement that does not significantly affect cell volume. The rate at which the measurements are repeated depends on the rate at which the cell is growing and the signal-to-noise ratio of the device. By increasing the sampling rate, the statistical significance of the measurement will improve. Both the VEM technique and the Coulter principle are independent of cell morthology and, with the appropriate design considerations, offer the sensitivity required to differentiate between linear and exponential cell growth in a single cell. The volume exclusion technique is disclosed in Gray et. al., “A New Method for Cell Volume Measurement Based on Volume Exclusion of a Fluorescent Dye,” Cytometry, Vol. 3, No. 6, Pages 428-434 (1983).

There are two major requirements that must be met in order for the system disclosed herein to operate with optimum performance First, a large number of cells must be maintained throughout multiple cell cycles. Microfluidic devices for circulating cells while maintaining order have not been demonstrated on a large scale. A goal of the present invention program will be to determine the maximum number of cells for which order can be maintained. While this is relatively straightforward to achieve for a single-layer microfluidic system, the system disclosed herein requires that cells travel through the SMR, interconnect holes in the silicon SMR substrate and several microfluidic valves. Thus, these components will need to be designed in a way to avoid dispersion in cell velocity. Cell order and velocity can be held constant by interspersing the cells with plugs of an immiscible material such as oil or air. If sized appropriately, these plugs would serve to compartmentalize the cells and maintain cell order as the cells and plugs travel through the various measurement modules, interconnect holes, and valves in the rotary channel. Conversely, cell order and velocity can also be maintained by encapsulating each cell inside an aqueous droplet within a continuous oil phase. Second, when a cell divides, it will be necessary to independently acquire measurements from each daughter cell. This requires that the cells be separated by a few hundred microns so they can be weighed individually by the SMR. To achieve the separation, shear force will be introduced to undivided cells with controllable pneumatic valves.

The contents of all of the references included herein and appended hereto are incorporated by reference herein in their entirety.

### REFERENCES

1. Burg, T. P., M. Godin, S. M. Knudsen, W. Shen, G. Carlson, J. S. Foster, K. Babcock, and S. R. Manalis, Weighing of biomolecules, single cells and single nanoparticles in fluid. Nature, 2007. 446(7139): p. 1066-9.
2. Conlon, I. and M. Raff, Size control in animal development. Cell, 1999. 96(2): p. 235-44.
3. Jorgensen, P. and M. Tyers, How cells coordinate growth and division. Curr Biol, 2004. 14(23): p. R1014-27.
4. Neufeld, T. P., A. F. de la Cruz, L. A. Johnston, and B. A. Edgar, Coordination of growth and cell division in the Drosophila wing. Cell, 1998. 93(7): p. 1183-93.
5. Iritani, B. M. and R. N. Eisenman, c-Myc enhances protein synthesis and cell size during B lymphocyte development. Proc Natl Acad Sci USA, 1999. 96(23): p. 13180-5.
6. Johnston, L. A., D. A. Prober, B. A. Edgar, R. N. Eisenman, and P. Gallant, Drosophila myc regulates cellular growth during development. Cell, 1999. 98(6): p. 779-90.
7. Dong, J., G. Feldmann, J. Huang, S. Wu, N. Zhang, S. A. Comerford, M. F. Gayyed, R. A. Anders, A. Maitra, and D. Pan, Elucidation of a universal size-control mechanism in Drosophila and mammals. Cell, 2007. 130(6): p. 1120-33.
8. Johnston, G. C., J. R. Pringle, and L. H. Hartwell, Coordination of growth with cell division in the yeast Saccharomyces cerevisiae. Exp Cell Res, 1977. 105(1): p. 79-98.
9. Nurse, P., Genetic control of cell size at cell division in yeast. Nature, 1975. 256(5518): p. 547-51.
10. Dolznig, H., F. Grebien, T. Sauer, H. Beug, and E. W. Mullner, Evidence for a size-sensing mechanism in animal cells. Nat Cell Biol, 2004. 6(9): p. 899-905.
11. Conlon, I. and M. Raff, Differences in the way a mammalian cell and yeast cells coordinate cell growth and cell-cycle progression. J Biol, 2003. 2(1): p. 7.
12. Pardee, A. B., A restriction point for control of normal animal cell proliferation. Proc Natl Acad Sci USA, 1974. 71(4): p. 1286-90.
13. Sherr, C. J. and J. M. Roberts, Living with or without cyclins and cyclin-dependent kinases. Genes Dev, 2004. 18(22): p. 2699-711.
14. Schorl, C. and J. M. Sedivy, Loss of protooncogene c-Myc function impedes GI phase progression both before and after the restriction point. Mol Biol Cell, 2003. 14(3): p. 823-35.
15. Ekholm, S. V., P. Zickert, S. I. Reed, and A. Zetterberg, Accumulation of cyclin E is not a prerequisite for passage through the restriction point. Mol Cell Biol, 2001. 21(9): p. 3256-65.
16. Martinsson, H. S., M. Starborg, F. Erlandsson, and A. Zetterberg, Single cell analysis of GI check points-the relationship between the restriction point and phosphorylation of pRb. Exp Cell Res, 2005. 305(2): p. 383-91
17. Cipollina, C., L. Alberghina, D. Porro, and M. Vai, SFPI is involved in cell size modulation in respirofermentative growth conditions. Yeast, 2005. 22(5): p. 385-99.
18. Di Talia, S., J. M. Skotheim, J. M. Bean, E. D. Siggia, and F. R. Cross, The effects of molecular noise and size control on variability in the budding yeast cell cycle. Nature, 2007. 448(7156): p. 947-51.
19. Elliott, S. G. and C. S. McLaughlin, Rate of macromolecular synthesis through the cell cycle of the yeast Saccharomyces cerevisiae. Proc Natl Acad Sci USA, 1978. 75(9): p. 4384-8.
20. Zong, W. X. and C. B. Thompson, Necrotic death as a cell fate. Genes Dev, 2006. 20(1): p. 1-15.
21. Wan, Y., X. Liu, and M. W. Kirschner, The anaphase promoting complex mediates TGF-beta signaling by targeting SnoNfor destruction. Mol Cell, 2001. 8(5): p. 1027-39.
22. Fu, A. Y., C. Spence, A. Scherer, F. H. Arnold, and S. R. Quake, A microfabricated fluorescence-activated cell sorter. Nature Biotechnology, 1999. 17: p. 1109-1111.
23. Grover, W. H., M. von Muhlen, Y. C. Weng, and S. R. Manalis, A microfluidic autosampler with true Teflon valves: Design and application to suspended microchannel resonator mass sensors. Proceedings of the 11^{th} International Conference on Miniaturized Systems for Chemistry and Life Sciences (MicroTAS), 2007.
24. Peer, D., P. Zhu, C. V. Carman, J. Lieberman, and M. Shimaoka, Selective gene silencing in activated leukocytes by targeting siRNAs to the integrin lymphocyte function-associated antigen-1. Proc Nat! Acad Sci USA, 2007. 104(10): p. 4095-100.
25. Unger, M. A., H. P. Chou, T. Thorsen, A. Scherer, and S. R. Quake, Monolithic Microfabricated Valves and Pumps by Multilayer Soft Lithography. Science. 2000. 288(5463): n. 113-1 16.

What is claimed is:

1. Microsystem for monitoring cell growth comprising:
microfluidic structure designed to allow cells to circulate therethrough, the microfluidic structure including modules to monitor at least one of mass, mass density and fluorescence of the cell.

2. Microsystem for cell sizing comprising:
a microfluidic rotary channel having a design that allows cells to circulate therethrough in single file;
a microfluidic pump to circulate the cells through the rotary channel;
a fluid delivery module to deliver nutrients and analytes within the rotary channel;
a fluorescence module for monitoring fluorescence from a cell;
a volume detection module for determining volume of the cells;
a mass detection module for determining mass of a cell; and
a mass density detection module for determining mass density of the cell.

3. The system of claim 2 wherein the mass detection module includes a suspended microchannel resonator.

4. The system of claim 2 wherein the volume detection module includes a cell volume measurement based on the exclusion of a fluorescent dye.

5. The system of claim 2 wherein the volume detection module includes a cell volume measurement based on the Coulter Principle.

6. The system of claim 1 further including means for moving the cells past a selected module multiple times.

7. Microsystem for cell sizing comprising:
a microfluidic rotary channel having a design that allows cells to circulate therethrough in single file;
a microfluidic pump to circulate the cells through the rotary channel; and
a mass detection module for determining mass of a cell.
